(19) United States
(12) Patent Application Publication
(10) Pub. No.: US 2023/0135784 A1
(43) Pub. Date: May 4, 2023

(54) USE OF VIRUS-LIKE PARTICLES INCLUDING TOXOPLASMA GONDII APICAL MEMBRANE ANTIGEN 1 FOR SERODIAGNOSING TOXOPLASMOSIS

(71) Applicant: University-Industry Cooperation Group of Kyung Hee University, Yongin-si (KR)

(72) Inventors: Fu-Shi Quan, Seoul (KR); Minju KIM, Seoul (KR)

(73) Assignee: University-Industry Cooperation Group of Kyung Hee University, Yongin-si (KR)

(21) Appl. No.: 17/969,632

(22) Filed: Oct. 19, 2022

(30) Foreign Application Priority Data
Oct. 28, 2021 (KR) 10-2021-0145421

(51) Int. Cl. G01N 33/569; C07K 14/45

(52) U.S. Cl. CPC G01N 33/56905; C07K 14/45

(57) ABSTRACT

The present disclosure relates to a use of virus-like particles including Toxoplasma gondii apical membrane antigen 1 for serodiagnosing toxoplasmosis, wherein an enzyme-linked immunosorbent assay with an infected serum was conducted using virus-like particles expressing Toxoplasma gondii AMA1, and higher sensitivity than the existing Toxoplasma gondii antigen was observed using virus-like particles, and no false diagnosis such as false negatives and false positives appeared after a reaction with a malaria-infected serum and thus high specificity was identified.

### CROSS-REFERENCE TO RELATED APPLICATION

This application claims the benefit of Korean Patent Application No. 10-2021-0145421 filed on Oct. 28, 2021, in the Korean Intellectual Property Office, the entire disclosure of which is incorporated herein by reference for all purposes.

### SEQUENCE LISTING

This application contains a Sequence Listing submitted via XML file and hereby incorporated by reference in its entirety. The Sequence Listing is named SEQ_2280-412.xml, created on Oct. 19, 2022 and 10,490 bytes in size.

### BACKGROUND

### 1. Field of the Invention

The present disclosure relates to a use of virus-like particles (VLPs) including Toxoplasma gondii apical membrane antigen 1 (AMA1) for serodiagnosing toxoplasmosis.

### 2. Description of the Related Art

Toxoplasma gondii is an obligate intracellular parasite, which is a protozoan pathogen that causes toxoplasmosis in humans and animals distributed worldwide. It is estimated that more than a third of the world's population is infected with Toxoplasma gondii, with 2 to 25% reported infection rate in Korea depending on the group.

Infection with Toxoplasma gondii in pregnant women may cause trophozoites of Toxoplasma gondii to pass through the placenta to infect the fetus. Early fetuses may be in danger of miscarriage or stillbirth due to infection with Toxoplasma gondii, and congenital malformations such as visual impairment, hydrocephalus, and mental retardation may occur in mid-late fetuses despite normal delivery. In addition, Toxoplasma gondii infecting healthy individuals disrupts cells in the immune system and reticuloendothelial system, causing diseases such as lymphadenitis, retinochoroiditis, and encephalomyelitis. Immune failure of a host activates cysts that proliferate in the brain, developing meningitis or retinochoroiditis as well.

On the other hand, pyrimethamine, a treatment for toxoplasmosis, is the most widely used, but a therapeutic effect is barely derived during pregnancy. Besides, spiramycin is used for prevention due to little therapeutic effects. In addition, sulfamethoxazole, a sulfa drug used in combination with pyrimethamine, may reduce the platelet count by causing bone marrow suppression, and side effects such as allergic reactions, kidney disorders, hematologic disorders, nausea, and vomiting may be accompanied by combined administration of folic acid.

Currently, there are many diagnostic methods for toxoplasmosis, and the most popular diagnostic method is an enzyme-linked immunosorbent assay (ELISA) using a tachyzoite lysate antigen (TLA)-antibody reaction. TLA used therefor is prepared by infecting mice with a protozoa, requiring sacrifices of many mice. Furthermore, there are disadvantages including low infection seroreactivity with the protozoan antigen used for diagnosing infection as well as false diagnosis such as false-negatives and false-positives. Therefore, there is a need to develop a more effective diagnostic method for toxoplasmosis.

### PRIOR ART DOCUMENT

### Patent Document

Korean Patent Registration No. 10-1256181 (Registered on Apr. 12, 2013)

### SUMMARY

### Problem to be Solved by the Invention

An object of the present disclosure is to provide a use of Toxoplasma gondii virus-like particles (VLPs) including influenza virus matrix protein 1 (M1) and Toxoplasma gondii apical membrane antigen 1 (AMA1) for diagnosing toxoplasmosis.

### Means for Solving the Problem

In order to achieve the above object, the present disclosure provides a composition for diagnosing toxoplasmosis, including, as an active ingredient, Toxoplasma gondii virus-like particles (VLPs) including influenza virus matrix protein 1 (M1) and Toxoplasma gondii apical membrane antigen 1 (AMA1).

In addition, the present disclosure provides a kit for diagnosing toxoplasmosis including the composition.

In addition, the present disclosure provides a method of providing information required for diagnosing toxoplasmosis, including (1) inducing an antigen-antibody reaction by bringing a sample isolated from a patient into contact with the composition for diagnosing toxoplasmosis; and (2) measuring a level of the antigen-antibody reaction to compare the level with that of a control sample.

### Effects of the Invention

The present disclosure relates to a use of virus-like particles including Toxoplasma gondii apical membrane antigen 1 for serodiagnosing toxoplasmosis. An enzyme-linked immunosorbent assay with an infected serum was conducted using virus-like particles expressing Toxoplasma gondii AMA1, and higher sensitivity than existing Toxoplasma gondii antigen was observed using virus-like particles. In addition, no false diagnosis such as false negatives and false positives appeared after a reaction with a malaria-infected serum and thus high specificity was also identified. A method of serodiagnosing toxoplasmosis using a virus-like particle protein of the present disclosure may be usefully applied for diagnosing toxoplasmosis owing to high sensitivity and specificity compared to existing diagnostic methods using Toxoplasma gondii antigen.

### BRIEF DESCRIPTION OF THE DRAWINGS

FIGS. 1 and 2 show results of identifying an antigen-serum reaction by orally infecting mice with 10, 50, 100, 150, and 300 cyst of Toxoplasma gondii (ME49 strain) using Toxoplasma gondii AMA1 virus-like particle protein antigen and then infecting serum thereafter, followed by collection at 1^{st}, 2^{nd}, 4^{th}, and 8^{th} week.

FIGS. 3 and 4 show results of identifying an antigen-serum reaction by orally infecting to mice with 5×10³, 1×10⁴, and 5×10⁴ Toxoplasma gondii (RH strain) using Toxoplasma gondii AMA1 virus-like particle protein antigen, and infecting serum thereafter, followed by collection at 1^{st}, 2^{nd}, 4^{th}, and 8 week.

FIG. 5 shows results of identifying a cross-reaction among infected sera of mice collected after infection with malaria (Plasmodium berghei), Toxoplasma gondii antigen, and Toxoplasma gondii AMA1 virus-like particle antigen.

FIG. 6 shows results of identifying an antigen-antibody reaction among serum of a patient infected with Toxoplasma gondii, Toxoplasma gondii antigen, and Toxoplasma gondii AMA1 VLPs.

### DETAILED DESCRIPTION

The present disclosure provides a composition for diagnosing toxoplasmosis, including, as an active ingredient, Toxoplasma gondii virus-like particles (VLPs) including influenza virus matrix protein 1 (M1) and Toxoplasma gondii apical membrane antigen 1 (AMA1).

In the present disclosure, “Toxoplasma gondii” is Apicomplexa in the subclass of coccidia. Toxoplasma gondii is an obligate intracellular parasite, a protozoan pathogen that causes toxoplasmosis in humans and animals distributed worldwide. Toxoplasma gondii mainly undergoes five stages of development, including oocyst, tachyzoite, bradyzoite, schizont, and gametocyte. Infection may occur by ingesting water or vegetables contaminated by oocysts in cat feces, the definitive host, or by ingesting Toxoplasma gondii present as cysts in meat of pig, sheep, and cattle, which are intermediate hosts. For example, the protozoa of Toxoplasma gondii may be Toxoplasma gondii ME49 strain, but is not limited thereto.

The term “virus-like particles (VLPs)” as used herein refers to non-infectious viral subunits with or without viral proteins. For example, the virus-like particle completely lacks a DNA or RNA genome, or a virus-like particle including a viral capsid protein may undergo spontaneous self-assembly.

In the present disclosure, “Toxoplasma gondii virus-like particle” induces an immune response specific to Toxoplasma gondii, and refers to a protein structure (particle) having a form similar to that of a virus.

The Toxoplasma gondii virus-like particle produces particles similar to virus as virus-derived structural proteins are assembled, and includes an epitope derived from the protozoa of Toxoplasma gondii, thereby inducing specific immune response against Toxoplasma gondii upon inoculation of the Toxoplasma gondii virus-like particles into a specific subject. As a specific example of the present disclosure, the protein structure may have a form on which an epitope derived from protozoa of Toxoplasma gondii is bound to the outside of a virus-derived structural protein.

The virus-derived structural protein (core protein) may be influenza virus matrix protein 1 (M1).

The term “influenza virus matrix protein 1 (M1)” as used herein refers to a structural protein of the influenza virus, and a matrix protein that constitutes a coat inside the fat layer, which is the envelope of the influenza virus. The influenza virus includes eight segmented negative-strand RNAs, surface proteins such as hemagglutinin (H) and neuraminidase (N), and proteins such as nucleoprotein (NP) matrix (M1), proton ion-channel protein (M2), polymerase acidic protein (PA), polymerase basic protein 2 (PB2), polymerase acidic protein (PA), and nonstructural protein 2 (NS2). In this case, matrix protein 1 functions as a linker between the core and the envelope by enclosing the outer structure with layers. The matrix protein 1 plays a crucial role in assembling the virus-like particles into a stable form in the production of virus-like particles.

The influenza virus matrix protein 1 may be derived from A/Puerto Rico/8/34, A/Bangkok/163/2000, A/AA/Huston/1945, A/Berlin/6/2006, A/Brandenburg/1/2006, A/Brevig Mission/1/1918, A/Chile/8885/2001, A/DaNang/DN311/2008, A/FLW/1951, A/FW/1/1950, A/Fiji/15899/83, A/Fort Monmouth/1-MA/1947, A/HaNoi/TX233/2008, A/Iowa/CEID23/2005, A/Malaysia/35164/2006, A/Managua/4086.04/2008, A/Texas/VR06-0502/2007, A/WSN/1933, A/Colorado/18/2011, A/Kentucky/04/2010, A/Maryland/28/2009, A/New Mexico/05/2012, A/Philippines/TMC10-135/2010, A/Singapore/GP4307/2010, A/Singapore/GP489/2010, A/Boston/14/2007, A/Brisbane/09/2006, A/Hong Kong/CUH34175/2002, A/Kyrgyzstan/WRAIR1256P/2008, A/Malaysia/12550/1997, A/Nanjing/1663/2010, A/Wyoming/08/2010, A/Berkeley/1/1968, A/Korea/426/1968, but is not limited thereto. Specifically, in an example embodiment of the present disclosure, M1 protein derived from A/Puerto Rico/8/34 was applied as a structural protein of virus-like particles.

The epitope derived from Toxoplasma gondii may be Toxoplasma gondii apical membrane antigen 1 (AMA1).

The term “epitope” as used herein refers to a basic element or minimum unit of recognition by each antibody or T cell receptor, and also refers to a specific domain, region, or molecular structure to which the antibody or T cell receptor binds. The epitope may be derived from Toxoplasma gondii and is not particularly limited as long as it may induce immunoactivity against the Toxoplasma gondii.

“Apical membrane antigen 1 (AMA1)” as used herein is an essential trace protein that plays an important role in host cell invasion. As a part of a moving junction (MJ) complex, apical membrane antigen 1 forms a circular structure between the plasma membrane of the apical tip of the parasite and the target host cell. During invasion, the moving junction complex moves from the anterior to the posterior of the parasite to be involved in the internalization of the parasite into the parasitophorous vacuole (PV). Accordingly, in the present disclosure, apical membrane antigen 1 is applied as a protein for the epitope.

Specifically, the influenza virus matrix protein 1 may include an amino acid sequence of SEQ ID NO: 1, and the apical membrane antigen 1 may include an amino acid sequence of SEQ ID NO: 2. In addition, the influenza virus matrix protein 1 or apical membrane antigen 1 may include a functional equivalent of the protein consisting of the amino acid sequence of SEQ ID NO: 1 or 2.

The term “functional equivalent” as used herein refers to a protein exhibiting a physiological activity substantially identical to that of the amino acid sequence of SEQ ID NO: 1 or 2, with sequence homology of at least 70% or higher, specifically 80% or higher, more specifically 90% or higher, most specifically 95% or higher with the amino acid sequence of SEQ ID NO: 1 or 2 as an outcome of addition, substitution or deletion of amino acids.

The term “substantially equivalent physiological activity” as used herein refers to an activity as a virus-like particle capable of inducing a specific immune response against Toxoplasma gondii due to structural and functional homology with the influenza virus matrix protein 1 or apical membrane antigen 1.

More specifically, the influenza virus matrix protein 1 may be encoded by a nucleic acid sequence of SEQ ID NO: 3, and the apical membrane antigen 1 may be encoded by a nucleic acid sequence of SEQ ID NO: 4. For example, the influenza virus matrix protein M1 may be a gene expressed by Genbank Accession No. ABO21712 or Genbank Accession No. EF467824. The apical membrane antigen 1 may be a gene expressed by Genbank Accession No. AF010264.1.

The virus-like particle acts as an antigen in a subject and may label T or B immune cells with the antigen through a reaction with antigen presenting cells such as dendritic cells.

In addition, the present disclosure provides a kit for diagnosing toxoplasmosis including the composition.

The diagnostic kit of the present disclosure may measure the antigen-antibody reaction via enzyme-linked immunosorbent assay (ELISA), radioimmunoassay (RIA), sandwich assay, Western blot on polyacrylamide gel, immunoblot assay, immunohistochemical staining, or surface enhanced Raman spectroscopy assay.

In this case, the diagnostic kit may include an immobilizer for immobilizing the Toxoplasma gondii virus-like particles; a secondary antibody conjugate conjugated with a chromophore that specifically binds to the Toxoplasma gondii virus-like particle; and a washing solution for a chromogenic substrate solution to be subjected to a color development reaction with the conjugate and an enzymatic reaction stop solution.

As the immobilizer for the antigen-antibody binding reaction, a nitrocellulose membrane, a PVDF membrane, a well plate synthesized with polyvinyl resin or polystyrene resin, and a slide glass made of glass may be used.

As the conjugate of the secondary antibody, a conventional color developer capable of carrying out a color development reaction may be desirable, and conjugates such as fluoresceins including horseradish peroxidase (HRP), alkaline phosphatase, colloid gold, poly L-lysine-fluorescein isothiocyanate (FITC), and Rhodamine-B-isothiocyanate (RITC) as well as a dye may be used.

In addition, the present disclosure provides a method of providing information required for diagnosing toxoplasmosis, including (1) inducing an antigen-antibody reaction by bringing a sample isolated from a patient into contact with the composition for diagnosing toxoplasmosis; and (2) measuring a level of the antigen-antibody reaction to compare the level with that of a control sample.

Preferably, the antigen-antibody reaction may be detected via enzyme-linked immunosorbent assay (ELISA), radioimmunoassay (RIA), sandwich assay, Western blot on polyacrylamide gel, immunoblot assay, immunohistochemical staining, or surface enhanced Raman spectroscopy assay, but is not limited thereto.

The term “sample isolated from a patient” as used herein refers to any substance of an organism, including blood, and the biological sample of the present disclosure includes tissues, cells, hair, oral tissues, oral cells, blood, lymph, bone marrow fluid, saliva, milk, urine, feces, ocular fluid, semen, brain extract, spinal fluid, joint fluid, ascites fluid, amniotic fluid, or tissue fluid, but is not limited thereto. Preferably, in the present disclosure, the sample may be blood which includes whole blood, plasma, and serum, and more preferably, the sample may be serum.

Hereinafter, the present disclosure will be described in detail with reference to example embodiments that do not limit the present disclosure. The following example embodiments of the present disclosure are merely for specifying the present disclosure, but are not intended to restrict or limit the scope of the present disclosure. Therefore, what may be easily inferred from the detailed description and example embodiments of the present disclosure by an expert in the art to which the present disclosure pertains is construed as belonging to the scope of the present disclosure.

### <Example 1> Preparation of Virus-Like Particles (VLPs) Including Toxoplasma gondii Apical Membrane Antigen 1 (AMA1)

In order to prepare a virus-like particle including influenza virus matrix protein 1 (M1) and apical membrane antigen 1 (AMA1) of Toxoplasma gondii protozoa, apical membrane antigen 1 of Toxoplasma gondii consisting of a nucleotide sequence of SEQ ID NO: 4 was selected as an antigen gene.

The sequence of the influenza matrix protein 1 (M1) gene used herein was the same as the M1 gene described in Korean Patent Application Publication No. 10-2019-0009691 (GenBank accession number: EF467824, 1027 bp).

Thereafter, the gene of influenza M1 protein was introduced into a pFastBac vector into which the AMA1 coding sequence was introduced so as to construct a recombinant plasmid. Here, whether the M1 and AMA1 genes introduced into the pFastBac vector were introduced properly was checked by DNA sequencing.

To prepare recombinant BaculoViruses (rBVs) expressing AMA1 and M1, DNA transfection was performed using cellfectin II (Invitrogen) and SF9 cells. Transformation using the pFastBac vector including AMA1 and M1 was performed by white/blue screening. Recombinant baculovirus was prepared according to a manual of a manufacturer through the Bacto-Bac expression system (Invitrogen).

Virus-like particles of Toxoplasma gondii were produced in SF9 insect cells co-infected with recombinant baculoviruses (rBVs) expressing AMA1 and M1. Virus-like particles in the supernatant were pelleted using high-speed centrifugation (30 min, 45,000×g).

Virus-like particles were resuspended overnight in phosphate buffered saline (PBS) at 4° C., harvested, and purified via a discontinuous sucrose gradient (20-30-60%) at 4° C. and 45,000×g for 1 hour. Protein concentration was determined by QuantiPro BCA Assay Kit (Sigma-Aldrich).

More details on the VLPs including Toxoplasma gondii AMA1 used in the present disclosure are described in Korean Patent Application No. 10-2020-0013081 (filing date: Feb. 4, 2020) previously filed by the present inventors.

### <Example 2> Verification of a Reaction Between Virus-Like Particle Protein Antigen and an Infected Serum in an Animal Model

1. Identification of an Antigen-Serum Reaction Using Toxoplasma gondii AMA1 Virus-Like Particle Protein Antigen

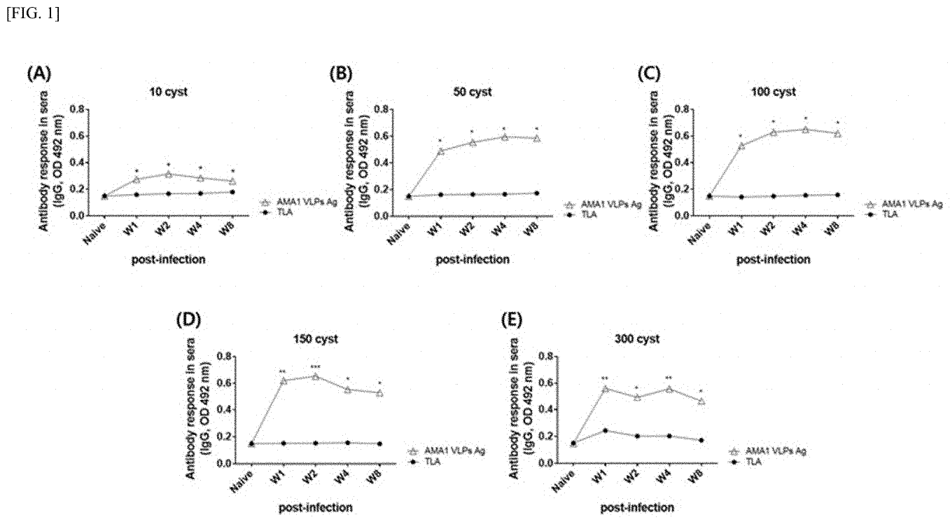

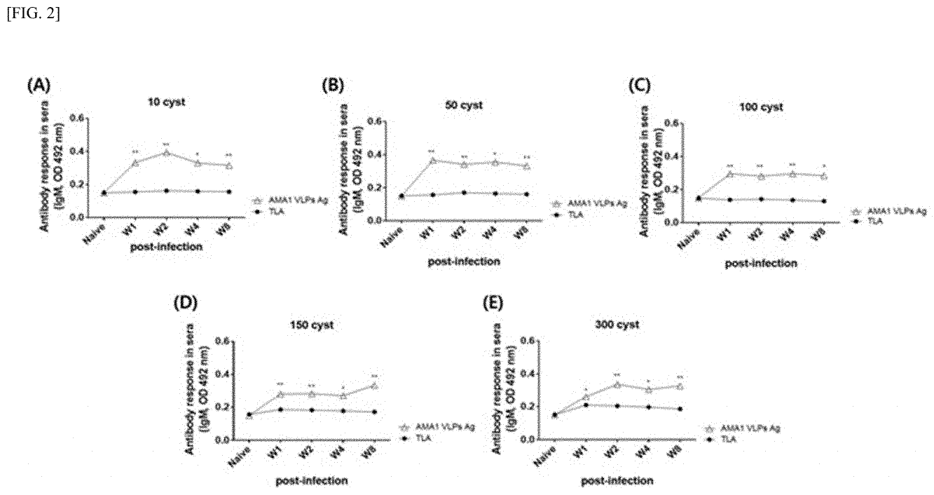

As shown in FIGS. 1 and 2, after oral infection with 10, 50, 100, 150, and 300 cyst of Toxoplasma gondii respectively, serum was collected at 1^{st}, 2^{nd}, 4^{th}, and 8^{th} week, followed by coating of 96 well immunoplates with Toxoplasma gondii lysate antigen (TLA) and Toxoplasma gondii AMA1 virus-like particles (VLPs) respectively. Thereafter, 100 μl/well of primary antibody was dispensed and reacted at 37° C. for 2 hours, wherein the primary antibody was obtained by 100 times diluting sera collected 1, 2, 4, and 8 weeks after oral infection of mice with 10, 50, 100, 150, and 300 cyst of Toxoplasma gondii (ME49 strain). 100 μl/well of secondary antibody obtained by 2000 times diluting goat anti-mouse IgG (FIG. 1) or IgM (FIG. 2) antibody was dispensed. After a reaction at 37° C. for 1 hour, 100 μl/well of substrate buffer added with H₂O₂ and O-Phenylenediamine (OPD) was dispensed and reacted. To prevent excessive reaction, 50 μl/well of sulfuric acid was dispensed to stop the reaction. OD values were measured using a microplate reader. After infection, all sera showed significantly higher responses with AMA1 VLPs than with TLA (*P<0.05, **P<0.01, ***P<0.001).

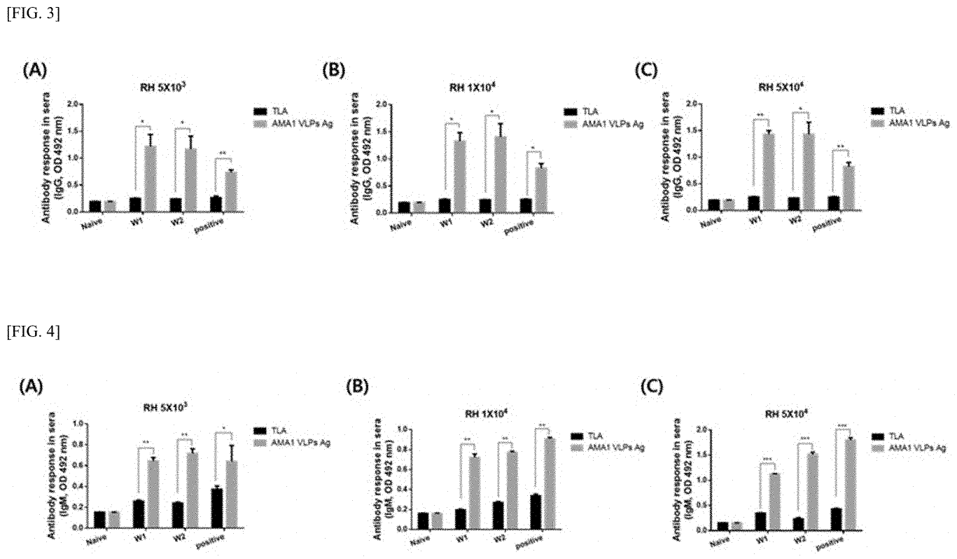

As shown in FIGS. 3 and 4, after oral infection with 5×10³, 1×10⁴, and 5×10⁴ Toxoplasma gondii (RH strain) respectively, sera were collected at 1^{st} and 2^{nd} week, followed by coating of 96 well immunoplates with Toxoplasma gondii lysate antigen (TLA) and Toxoplasma gondii AMA1 virus-like particles (VLPs) respectively. 100 μl/well of primary antibody was dispensed and reacted at 37° C. for 2 hours, wherein the primary antibody used herein was obtained by 100 times diluting the sera obtained 1 and 2 weeks after oral infection with 5×10³, 1×10⁴, and 5×10⁴ Toxoplasma gondii (RH strain). 100 μl/well of secondary antibody obtained by 2000 times diluting goat anti-mouse IgG (FIG. 3) or IgM (FIG. 4) antibody was dispensed, and a reaction was carried out at 37° C. for 1 hour. Thereafter, 100 μl/well of substrate buffer added with H₂O₂ and O-phenylenediamine (OPD) was dispensed and reacted. To prevent excessive reaction, 50 μl/well of sulfuric acid was dispensed to stop the reaction. OD values were measured using a microplate reader. After infection, all sera showed significantly higher responses with AMA1 VLPs than with TLA (*P<0.05, **P<0.01, ***P<0.001).

2. Identification of a Cross-Reaction Among Infected Sera of Mice Collected after Infection with Malaria (Plasmodium berghei), Toxoplasma gondii Antigen, and Toxoplasma gondii AMA1 Virus-Like Particle Antigen

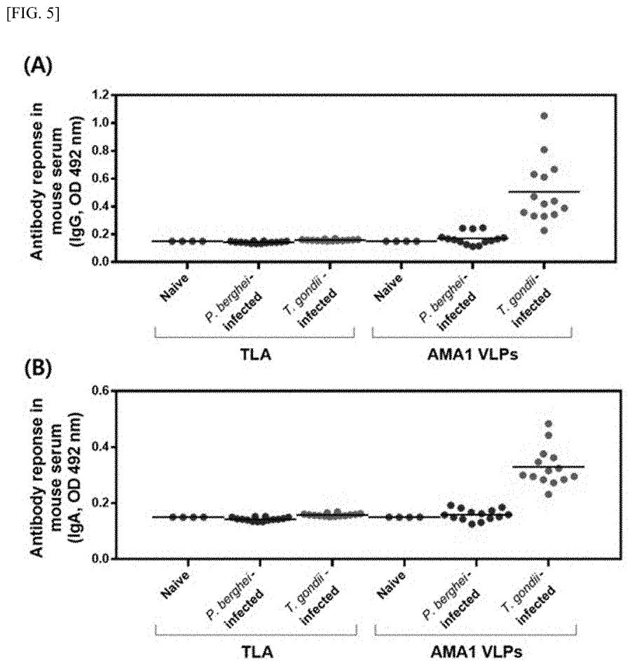

To observe the cross-reaction of the Toxoplasma gondii AMA1 gene, the serum obtained by intramuscular inoculation of Plasmodium berghei protozoa into mice, Toxoplasma gondii lysate antigen (TLA) and Toxoplasma gondii AMA1 VLP antigen were subjected to an antigen-antibody reaction, and an IgG antibody titer (FIG. 5A) and an IgA antibody titer (FIG. 5B) were observed (FIG. 5). Toxoplasma gondii lysate antigen (TLA) and Toxoplasma gondii AMA1 virus-like particles (VLPs) were coated on 96 well immunoplates, respectively, and 100 μl/well of primary antibody obtained by 100 times diluting Toxoplasma gondii-infected serum and malaria-infected serum was dispensed and reacted at 37° C. for 2 hours. 100 μl/well of secondary antibody obtained by 2000 times diluting goat anti-mouse IgG or IgM antibody was dispensed, and a reaction was carried out at 37° C. for 1 hour. Thereafter, 100 μl/well of substrate buffer added with H₂O₂ and O-phenylenediamine (OPD) was dispensed and reacted. To prevent excessive reaction, 50 μl/well of sulfuric acid was dispensed to stop the reaction. OD values were measured using a microplate reader. No cross-reaction was observed with the malaria-infected sera, and higher response was detected in the Toxoplasma gondii-infected sera as in the previous experiments. Thereby, the sensitivity and specificity of Toxoplasma gondii AMA1 VLP antigen were identified.

3. Identification of an Antigen-Antibody Reaction Among Serum of a Toxoplasma gondii-Infected Patient, Toxoplasma gondii Antigen, and Toxoplasmosis AMA1 VLPs

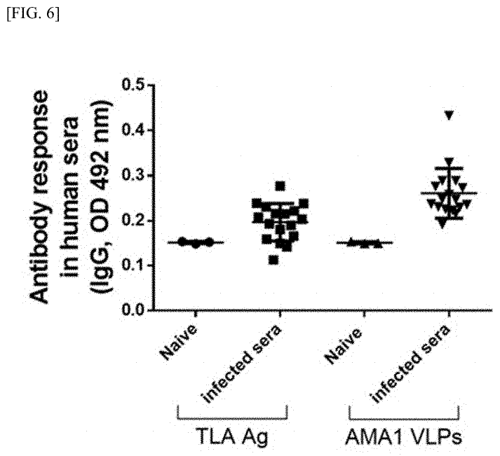

Sera of Toxoplasma gondii-infected patients were obtained from Chungnam National University and Seoul National University, and IgG antibody titer was observed by carrying out an antigen-antibody reaction between Toxoplasma gondii antigen and Toxoplasma gondii AMA1 VLPs, respectively (FIG. 6). Toxoplasma gondii lysate antigen (TLA) and Toxoplasma gondii AMA1 virus-like particles (VLPs) were coated on 96 well immunoplates respectively, and 100 μl/well of primary antibody obtained by 100 times diluting Toxoplasma gondii-infected serum was dispensed and reacted at 37° C. for 2 hours. 100 μl/well of secondary antibody obtained by 2000 times diluting goat anti-mouse IgG antibody was dispensed, and a reaction was carried out at 37° C. for 1 hour. Thereafter, 100 μl/well of substrate buffer added with H₂O₂ and O-phenylenediamine (OPD) was dispensed and reacted. To prevent excessive reaction, 50 μl/well of sulfuric acid was dispensed to stop the reaction. OD values were measured using a microplate reader. Though it was the serum of an infected patient, the results showed that little difference was found in Toxoplasma gondii antigen compared to the uninfected serum (naïve). However, different results from the uninfected serum were derived in all samples for Toxoplasma gondii AMA1 VLP antigen. Thereby, it was found that the false-negative and false-positive results that appeared in existing diagnostic methods could be removed by the diagnostic method using the Toxoplasma gondii AMA1 VLP antigen.

What is claimed is:

1. A method of diagnosing toxoplasmosis, comprising:
(1) inducing an antigen-antibody reaction by bringing a sample isolated from a patient into contact with a composition for diagnosing toxoplasmosis,
wherein the composition comprises, as an active ingredient, Toxoplasma gondii virus-like particles (VLPs) comprising influenza virus matrix protein 1 (M1) and Toxoplasma gondii apical membrane antigen 1 (AMA1); and
(2) measuring a level of the antigen-antibody reaction to compare the level with that of a control sample.

2. The method of claim 1, wherein the sample is blood or serum.

3. The method of claim 1, wherein the level of the antigen-antibody reaction is identified by enzyme-linked immunosorbent assay (ELISA), radioimmunoassay (RIA), sandwich assay, Western blot on polyacrylamide gel, immunoblot assay, immunohistochemical staining, or surface enhanced Raman spectroscopy assay.

4. The method of claim 1, wherein the M1 comprises an amino acid sequence of SEQ ID NO: 1, and the AMA1 comprises an amino acid sequence of SEQ ID NO: 2.

5. The method of claim 1, wherein the M1 is encoded by a nucleic acid sequence of SEQ ID NO: 3, and the AMA1 is encoded by a nucleic acid sequence of SEQ ID NO: 4.

6. A kit for diagnosing toxoplasmosis, comprising the composition based on claim 1.

7. A kit for diagnosing toxoplasmosis, comprising the composition based on claim 4.

8. A kit for diagnosing toxoplasmosis, comprising the composition based on claim 5.


SEQUENCE LISTING (4 sequences)

SEQ ID NO: 1 - protein, 618 aa, synthetic construct
  REGION 1..618: note = M1
MKISIVAFPLLMIALRSKSTNAHKTNNLEAQINYGIINNYNELLKVAKCQYCLTTTNPVEEENCDEIMEECRGLLSNKDLGFLLKAITDESMHNKSQYIHGKHSNTLRRIIKVLEAQKKNIESVKNIVRDIKKSGNTQLRSSGTSISDLDKLNTSIKNIKKGFQFLNDNYSTINKHINIPSGDMNKIYKRIVNTNNFDGLSKSQEKICSNEDDTNVGIDDIIKSSVQDIFDEGENIMNVVKTVLVQESDGIGNELAGLIEKGKEIGEQIVNIEGLLSPKNGLFSGGLPSLNKLYEFTSNLSSYEYLLVKLKDSIISKLKDILLRLLYKSYITYRKNKSIELGEEEIPMVSKDEYLDELKKGVIQLSMKLLYSKIKRLLIKIKNKMSRKKKTENIPDPLPVESSIYDYDDDDAIVDDTADDDAADDDVADNDKFISFNKSSSHMKLFRGILPQKKSIVSTIDKMISEIDLYEQGLYTDTHADYENDEILSTVEGMDETESDEAELSNECVQKIIDENIAVEAINNLLKVDESAVEERENVNDSENKSNNSSIDIEKGASTPSDISEIPNIIKKIVIYVIKERIYDLAEELSDNKLEDESKTSTPSNNIILEDTPAPNQA

SEQ ID NO: 2 - protein, 541 aa, synthetic construct
  REGION 1..541: note = AMA1
MGLVGVQVLLVLVADCTIFASGLSSSTRSRESQTLSASTSGNPFQANVEMKTFMERFNLTHHHQSGIYVDLGQDKEVDGTLYREPAGLCPIWGKHIELQQPDRLPYRNNFLEDVPTEKEYKQSGNPLPGGFNLNFVTPSGQRISPFPMELLEKNSNIKASTDLGRCAEFAFKTVAMDKNNKATKYRYPFVYDSKKRLCHILYVSMQLMEGKKYCSVKGEPPDLTWYCFKPRKSVTENHHLIYGSAYVGENPDAFISKCPNQALRGYRFGVWKKGRCLDYTELTDTVIERVESKAQCWVKTFENDGVASDQPHTYPLTSQASWNDWWPLHQSDQPHSGGVGRNYGFYYVDTTGEGKCALSDQVPDCLVSDSAAVSYTAAGSLSEETPNFIIPSNPSVTPPTPETALQCTADKFPDSFGACDVQACKRQKTSCVGGQIQSTSVDCTADEQNECGSNTALIAGLAVGGVLLLALLGGGCYFAKRLDRNKGVQAAHHEHEFQSDRGARKKRPSDLMQEAEPSFWDEAEENIEQDGETHVMVEGDY

SEQ ID NO: 3 - DNA, 1857 bp, synthetic construct
  misc_feature 1..1857: note = M1
  source 1..1857: mol_type = other DNA
atgaagataagtatcgtggcattccctttacttatgattgctctcagaagcaaatctaccaatgctcataaaacaaataatttggaagcccaaattaattatggtattataaataattataacgaattgttaaaagtagcaaaatgccaatattgcttaactaccaccaatcctgttgaagaagaaaattgtgatgaaataatggaagaatgtagaggattgttaagcaataaagaccttggatttttattaaaagccataacagatgagtctatgcataataaatctcaatatattcatggaaaacatagtaatacattaagaagaattattaaagttttagaagcacaaaaaaaaaatattgaatcagtaaaaaatattgtacgtgatattaaaaaaagtggtaatacacagttaagaagtagtggtacaagtatttcagatttagataaattaaacacaagcattaaaaatataaaaaaaggtttccaatttttaaatgataattacagcacaattaataaacacataaatataccaagtggtgatatgaataaaatttataaaagaatagtaaacacaaataattttgatgggttatcaaaatcacaagaaaaaatttgttcaaatgaagacgacaccaatgtaggtattgatgatattataaaatcaagtgttcaagatatttttgatgagggagaaaatataatgaatgtagttaaaactgttttagtacaagaaagtgatggaattggaaatgaattagcagggttaattgaaaaaggaaaagaaatcggagaacaaattgtaaatattgaaggattgttatctccaaaaaatggattatttagtggcggtttaccatcattaaataagttgtatgaatttacaagtaatttatcatcttatgaatatttgttagttaagctcaaagattcaataatatcaaaattaaaagacatattattaagattgttatataaatcatatataacttacagaaaaaataaatcaattgagttaggagaagaagaaataccaatggttagcaaggatgaatatttagatgaattaaaaaaaggtgtaatacaattaagtatgaaattattatatagcaaaatcaaaaggttattaattaaaattaaaaacaaaatgtcccgtaaaaagaaaacggaaaatattcctgacccattaccagttgaatcctcaatttatgattatgatgatgacgatgcaattgtcgacgatacagctgatgatgatgcagctgatgatgatgtagctgataatgataaatttatttcatttaataaatcctcgtcacatatgaaattattcagaggtattttgccccagaaaaaatctattgtatctaccatcgataagatgattagtgaaatcgatttatatgaacaaggattatatactgatacacatgcagattatgaaaacgatgaaatcttatctactgtcgaaggtatggatgaaacagaatctgatgaagctgaattgtcaaatgagtgtgttcagaaaattatcgatgaaaacattgccgtagaagctatcaacaatttacttaaagtcgatgaatctgcagtagaggaaagggaaaatgtaaatgattcagaaaataaatcaaataattcatccattgacattgaaaagggtgctagtactcctagtgacatttcagaaattcctaacatcataaaaaaaatcgttatatatgttataaaagaaagaatatatgatttagcagaagaattatcagacaataaactcgaggatgaatctaaaacatcaacaccttcaaacaatataatcctagaagatacaccagctccaaaccaagcataa

SEQ ID NO: 4 - DNA, 1626 bp, synthetic construct
  misc_feature 1..1626: note = AMA1
  source 1..1626: mol_type = other DNA
atggggctcgtgggcgtacaagttttgctggttcttgtggcggattgcaccatattcgcatcgggactcagctcaagcacaaggtctcgcgagtcgcagacgctgagtgctagcacgtcggggaatccctttcaggcaaatgtagagatgaaaaccttcatggaaagattcaacctaactcatcatcatcagtctggtatttacgtcgaccttggacaagacaaggaagttgatggcacattataccgggagcctgcggggttgtgtcccatttggggaaagcacatcgaactccagcagccggaccggcttccgtaccgtaacaacttcttggaagatgttccgactgaaaaagaatacaaacagtcagggaatcctttgcccggaggcttcaacttgaatttcgtgacgcctagcgggcagcgaatttcaccatttccgatggaacttcttgaaaaaaatagcaacatcaaggcgagtacggatcttgggaggtgcgccgagtttgcctttaagacggtcgctatggataaaaacaataaggcgacgaagtaccgttacccatttgtttatgactccaagaagcgactgtgccacatcctctacgtatcgatgcagctgatggagggtaaaaagtactgttcagtcaagggcgaacctccagatctcacatggtattgcttcaagccccgaaagagtgttacggagaatcatcatctcatctacggatcggcctatgttggagagaacccagatgcgttcatcagtaaatgcccaaatcaagctcttcgcgggtacaggttcggtgtttggaagaaaggccgttgcctcgactacactgaattgaccgacactgtgatagaacgtgttgagtcaaaggcacagtgctgggtgaaaacctttgaaaacgacggggtcgcgagtgaccaaccccatacgtatccactgacgtcgcaagcatcatggaacgattggtggcctctccaccagagtgaccaacctcactcaggtggcgttgggcgtaattacggtttctactacgtggacacgactggagagggcaagtgtgcactctctgaccaggtacccgactgcctggtgtcggattctgccgccgtgtcgtatacagcagcggggagtttgtctgaagagacgccgaatttcataattccgtcaaatccctctgttactccgccaacgcccgagacggcacttcagtgcacggccgacaagttccccgactctttcggtgcctgcgacgttcaagcctgtaaaagacagaagacgtcctgcgttggcggacagattcaaagtactagcgtcgactgcaccgcggacgaacaaaatgaatgtggctctaacactgcgttgatcgctggactcgccgtaggaggggttctgctgttggctcttctaggaggaggctgctacttcgcgaagaggttggacagaaacaaaggcgtccaggcggctcatcatgaacatgagtttcagtcagacagaggtgctcgaaaaaagaggccaagcgatctcatgcaagaggctgaaccgtcgttttgggatgaggcagaggagaacattgaacaagatggggaaacacatgttatggtcgagggggattactag